(19) United States
(12) Patent Application Publication
(10) Pub. No.: US 2007/0237737 A1
(43) Pub. Date: Oct. 11, 2007

(54) Formulation and method for a skin treatment for animals

(75) Inventors: Thomas W. Dann, Oldsmar, FL (US); Michael C. Fleck, Bradendon, FL (US)

Correspondence Address: DLA PIPER US LLP, 4365 EXECUTIVE DRIVE, SUITE 1100, SAN DIEGO, CA, 92121-2133, US

(73) Assignee: Epidermapet, LLC, Bradenton, FL (US)

(21) Appl. No.: 11/402,503

(22) Filed: Apr. 11, 2006

(51) Int. Cl. A61K 8/97; A61K 36/534; A61K 31/4172; A61K 31/198; A61K 36/61

(52) U.S. Cl. 424/74; 424/747; 424/769; 514/400; 514/561

(57) ABSTRACT

An aqueous composition for treating the skin of an animal is provided, the composition including a moisturizer, an anti-itch agent, a skin conditioner, a hair conditioner, a cleansing agent, and an aromatherapy agent. A method for preparing such a composition as well as a method for using the composition for improving the skin condition of a domestic animal such as a dog, a cat, or a horse are also provided.

### FIELD OF THE INVENTION

The present invention relates generally to animal skin care products and more specifically to methods of using such products for topical treatment of pets in need of improvement of quality of skin.

### BACKGROUND

Many domestic animals, including dogs and cats, frequently suffer from dry, oily, itchy, or flaky skin, or from similar skin defects, maladies, or imperfections. The skin of the animals is also often malodorous. Previously, many products, such as shampoos, fragrances and the like have been developed for treating at least some of such conditions. Mostly, such products were of a cosmetic nature and failed to effectively treat the underlying sources of such maladies. At best, only limited and temporary relief was achieved using such traditional products.

The nature of animal skin differs substantially from that of human skin in many respects. More specifically, human skin is quite acidic having a pH of about 5.2, while the animal skin is essentially neutral and has a pH of about 7.0. In addition, animal skin is much thicker than human skin and is covered with a layer of hair that is also much thicker. Accordingly, the methods of treatment of human skin having the above-described problems cannot be easily adopted for the treatment of animal skin.

It is very desirable to have a product that can remedy all or many of the above-described problems and to reduce dandruff, neutralize and/or eliminate oil from the skin, soften and brighten the hair coat, moisturize the skin, repel insects, de-odorize the skin and add a pleasant fragrance to it. Currently, such a product has not been provided. The present application provides the formulations that achieve these goals and describes the methods of using such compositions.

### SUMMARY

The present invention provides an aqueous composition for treating a skin of an animal, the composition including a moisturizer, an anti-itch agent, a skin conditioner, a hair conditioner, a cleansing agent, and an aromatherapy agent. The composition can further optionally include an insect repellant and/or a viscosity decreasing agent.

The present invention further provides a method for preparing such a composition as well as a method for using the composition for improving the skin of a domestic animal such as a dog, a cat, or a horse.

### BRIEF DESCRIPTION OF DRAWINGS

FIGS. 1-3 demonstrate schematically a method of treating a domestic animal according to an embodiment of the present invention.

### DETAILED DESCRIPTION

The following are the definitions of some terms as used in the present application. If the terms and definitions provided below are inconsistent in any way with their generally accepted meanings, then, only to the extent of such potential inconsistencies, the meanings provided below prevail.

The term “dry skin” refers to a skin condition that is characterized by the visible presence of white, flaky debris comparable to the human scalp condition known as dandruff. The term “flaky skin” refers to a skin condition when a relatively large amount of such debris is present.

The term “oily skin” refers to a skin condition that is characterized by the presence of the excessive amount of body oil on the animal's skin and is typically the result of dehydrated skin and is often also accompanied by pungent odors.

The term “itchy skin” refers to a skin condition leading to an irritating skin sensation. Such sensation causes an animal to scratch itself.

The term “a moisturizer” refers to a substance designed to make softer and more pliable the epidermis (i.e., the external layers of the skin), by increasing the extent of its hydration, allowing the addition of water to the epidermis, or the retention of water by the epidermis, or both.

The term “an anti-itch agent” refers to a substance designed to eliminate, reduce or relieve the “itchy” skin condition defined above.

The terms “a skin conditioner” “a hair conditioner” refer to a substance that improves the quality of the skin or hair, respectively, such as improving softness, manageability and luster.

The term “a cleansing agent” refers to a substance having cleaning power and capable of removing dirt, contaminants, and impurities from the skin and hair of an animal.

The term “an aromatherapy agent” refers to a substance providing fragrant, natural, botanical oils capable of rendering positive therapeutic effect when applied to an animal. Such oils can be obtained from various plants, leaves, bark, roots, seeds, resins and flowers.

The term “a viscosity decreasing agent” is defined as a substance substantially reducing viscosity of a liquid composition compared to the viscosity of the composition in the absence of such substance.

The term “an insect repellent” refers to a substance applied to skin of an animal which discourage insects and other arthropods from landing or climbing on the skin.

The term “topically” refers to direct application of a substance on the skin or hair of an animal.

The term “spraying” refers to a process of the application of a liquid in the form of small particles ejected from a spraying device.

The term “aerosol” refers to a suspension of liquid particles in air.

The term “galactoarabinan” refers to a naturally occurring functional polysaccharide, which can be extracted from North American larch trees, which typically grow in Minnesota and the Northwest.

The term “urea” refers to a chemical compound having the formula (I):

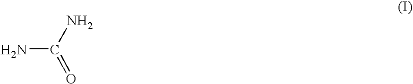

The term “sodium lactate” refers to a chemical compound having the formula (II):

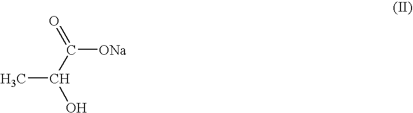

The term “an amino acid” refers to any compound that contains both amino and carboxylic acid functional groups

The term “ethoxydiglycol” refers to a chemical compound having the formula (III):
HO [symbol] CH₂ [symbol] ₂O [symbol] CH₂ [symbol] ₂O—CH₂—CH₃ (III)

The terms “glycine” and “histidine” refer to compounds shown by formulae (IV) and (V), respectively:

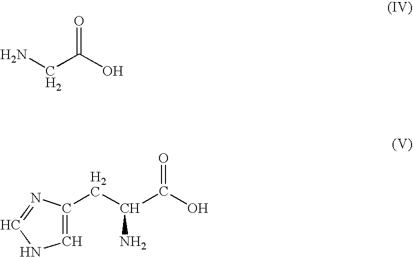

The terms “peppermint” and “spearmint” refer to oil extracted from the dried leaves and flowering tops of the plant mentha piperita, and mentha spicata, respectively.

The term “tea tree oil” refers to oil extracted from Australian tree melaleuca alternifolia.

The term “allantoin” refers to a derivative of urea having an imidazole-derived moiety. Allantoin is a crystallizable product of oxidation of uric acid having the formula (VI) shown below. Allanotoin can be found in allantoic and amniotic fluids, in fetal urine, and in many plants. Allantoin can be also obtained synthetically.

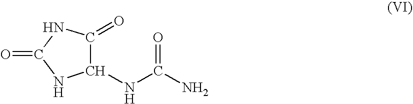

The term “pennyroyal” refers to oil extracted from any of various mint plants, especially mentha pulegium.

The term “lemongrass” refers to oil extracted from the tropical grass cymbopogon citratus native to southern India and Ceylon.

The term “cetrimonium chloride” refers to a chemical compound having the formula (VII):

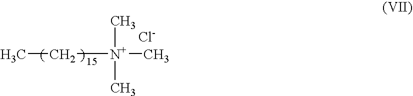

The term “sodium lauroyl lactylate” refers to a chemical compound having the formula (VIII):

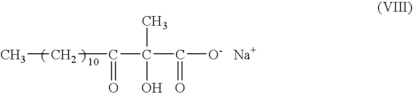

The term “lavender” refers to oil extracted from various species of the plant lavendula latifolia.

The term “cedar oil” refers to oil extracted from various species of cedar trees.

The term “patchouli” refers to oil extracted from a small southeast Asian shrub pogostemon cablin of the mint family.

The term “poly(ethylene glycol) methyl glucose trioleate” refers to a compound that is an adduct of methylglucose trioleate having the structure (IX) and poly(ethylene glycol):

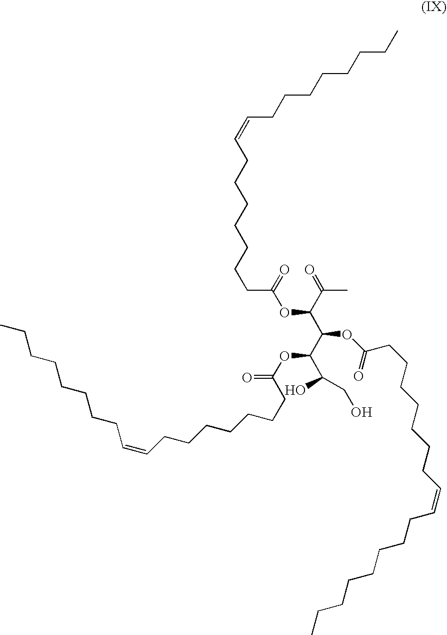

According to an embodiment of the present invention, an aqueous composition is provided for treating animals having dry and/or itchy skin, or related skin defects and imperfections. The composition can include at least one moisturizer, at least one anti-itch agent, at least one skin conditioner, at least one hair conditioner, at least one cleansing agent, and at least one aromatherapy agent. Optionally, the composition can additionally include at least one viscosity decreasing agent and/or at least one insect repellent. The overall composition can contain a total of between about 10 and 15 mass % of all the above-mentioned components combined and the balance of water. Optionally, the composition can also be formulated using any commonly used buffer system, if maintaining a certain level of pH is necessary.

The total concentration of the moisturizer(s) in the composition can be between about 0.1 and 8.0 mass % of the total composition. One example of a moisturizer that can be used is galactoarabinan, which can reduce transepidermal water loss. The concentration of galactoarabinan in the total composition, regardless of the presence or absence of other moisturizer(s), can be between about 0.1 and 0.9 mass %, such as between about 0.3 and 0.7 mass %, for example, about 0.5 mass %.

Other non-limiting examples of moisturizers that can be used include urea (which is a humectant), sodium lactate (also a humectant), and some amino acids, such as glycine or histidine. If urea is used as a moisturizer, its contents in the total composition, regardless of the presence or absence of other moisturizer(s), can be between about 1.0 and 5.0 mass %, such as between about 2.0 and 4.0 mass %, for example, about 4.0 mass %. If sodium lactate is used as a moisturizer, its contents in the total composition, regardless of the presence or absence of other moisturizer(s), can be between about 0.5 and 2.0 mass %, such as between about 1.0 and 2.0 mass %, for example, about 1.0 mass %.

The total concentration of the anti-itch agent(s) in the composition can be between about 0.1 and 2.0 mass % of total content. The anti-itch agent(s) that can be used can also serve as antioxidant(s). One example of an anti-itch agent that can be used is tea tree oil, the contents of which in the total composition, regardless of the presence or absence of other anti-itch agent(s), can be between about 0.1 and 0.9 mass %, such as between about 0.3 and 0.7 mass %, for example, about 0.5 mass %.

The total concentration of the skin conditioner(s) in the composition can be between about 0.5 and 2.5 mass % of total content. One example of a skin conditioner that can be used is allantoin, the contents of which in the total composition, regardless of the presence or absence of other skin conditioner(s), can be between about 0.01 and 0.9 mass %, such as between about 0.01 and 0.5 mass %, for example, about 0.02 mass %.

Other non-limiting examples of skin conditioners that can be used include sodium lactate, pennyroyal, lemongrass, and urea. If sodium lactate is used as a skin conditioner, its contents in the total composition, regardless of the presence or absence of other skin conditioner(s), can be between about 0.5 and 2.0 mass %, such as between about 1.0 and 1.5 mass %, for example, about 1.0 mass %. If pennyroyal or lemongrass is used as a skin conditioner, contents of either in the total composition, regardless of the presence or absence of other skin conditioner(s), can be between about 0.05 and 0.9 mass %, such as between about 0.01 and 0.5 mass %, for example, about 0.1 mass %.

The total concentration of the hair conditioner(s) in the composition can be between about 1.0 and 5.0 mass % of total content, such as between about 1.5 and 4.0 mass %, for example, about 2.0 mass %. One example of a hair conditioner that can be used is cetrimonium chloride. Non-limiting examples of other acceptable hair conditioners include sodium lauroyl lactylate and amino acids, such as glycine or histidine.

Non-limiting examples of cleansing agents that can be used include a derivative of poly(ethylene glycol) (PEG), such as PEG-120 methyl glucose trioleate, and octoxynol-9 (PEG-9 octyl phenyl ether), the concentration of each of which in the total composition can be between about 0.1 and 0.9 mass %, such as between about 0.2 and 0.7 mass %, for example, about 0.2 mass %.

The total concentration of the aromatherapy agent(s) in the composition can be between about 0.01 and 0.9 mass % of total content. Examples of aromatherapy agents that can be used include lavender and cedar oil. If lavender is used as an aromatherapy agent, its contents in the total composition, regardless of the presence or absence of other aromatherapy agent(s), can be between about 0.1 and 0.9 mass %, such as between about 0.2 and 0.5 mass %, for example, about 0.25 mass %. If cedar oil is used as an aromatherapy agent, contents of either in the total composition, regardless of the presence or absence of other aromatherapy agent (s), can be between about 0.01 and 0.9 mass %, such as between about 0.05 and 0.5 mass %, for example, about 0.05 mass %.

If insect repellent(s) are used, the total concentration of the insect repellent(s) in the composition can be between about 0.1 and 0.9 mass % of total content, such as between about 0.3 and 0.7 mass %, for example, about 0.5 mass %. Non-limiting examples of insect repellents that can be used include lemongrass, peppermint, spearmint, pennyroyal, and patchouli.

If viscosity decreasing agent(s) are used, an example viscosity decreasing agent that can be used is ethoxydiglycol, which can be present at the concentration of between about 1.0 and 5.0 mass % of total composition, such as between about 1.5 and 4.5 mass %, for example, about 4.0 mass %.

Any composition satisfying the above-described requirements can be prepared using common formulating techniques known to those having ordinary skill in the art. For example, the above-described components can be mixed with one another, followed by adding water, to form an aqueous composition, e.g., by employing rapid stirring. Alternatively, each component, in a separate container, can be preliminary dissolved in water, or otherwise mixed with water resulting in a plurality of water-based systems, each contained in a separate container. The contents of all the containers can then be combined, e.g., by stirring or shaking, to form the final composition.

If desired, those having ordinary skill in the art can design other methods of mixing the components forming the composition. Regardless of the method of mixing that is selected, those having ordinary skill in the art will provide such quantities of each component so that the concentration of each of the components in the composition satisfies the above-described limits.

A method for the treatment of an animal is further provided. A composition can be prepared according to a procedure described above. The composition can be then applied topically onto the skin of an animal that needs the improvement of its skin condition. Various methods can be used for applying the composition onto the skin of an animal. For example, the composition can be sprayed using a conventional hand-operated pump. Alternatively, the composition can be formulated to form an aerosol using commonly known methods for aerosol preparation. The composition can be also formulated as a solution or a shampoo. Those having ordinary skill in the art can devise other methods for applying the composition.

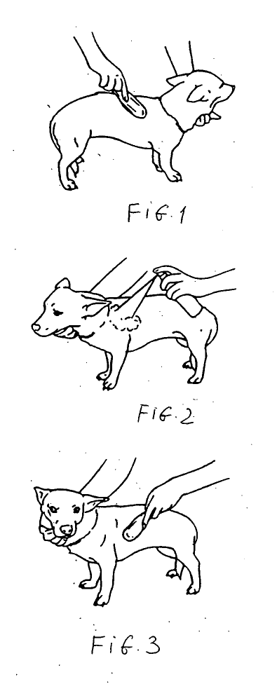

Referring now to FIGS. 1-3, one exemplary procedure that can be utilized for the treatment of a dog can be briefly described as follows. A suitable brush can be used for the procedure below, such as a two-sided brush that has metal bristles on one side and nylon-type bristles on the other. Using such or similar brush, the hair coat and skin of a dog can be energetically brushed using the metal side of the brush (FIG. 1). The duration of brushing can be at least 30 seconds.

Following the brushing procedure, the composition can be sprayed onto the entire body of the dog (FIG. 2). A hand-pump can be used for spraying. Alternatively, the composition can be applied using an aerosol generator, such as an aerosol can. At least 10 sprays can be used. The heaviest spraying can be applied over the back and sides of the dog. Fewer sprays can be applied for a smaller dog, and more sprays can be used if the dog is larger. The product should not be sprayed into the dog's eyes. Instead, the product can be applied to the brush first, followed by gentle brushing over the head and face.

When the product has been sprayed on, the product can then be brushed into the hair coat and skin of the dog using the metal side of the brush (FIG. 3). The duration of brushing can be at least 30 seconds. Then, the brush head can be reversed and the hair coat can be gently smoothed over. The soft side of the brush can be used for brushing the head and underside of the dog. If the dog's skin is red and sensitive, the softer side of the brush can be used. The entire procedure can be repeated daily.

A variety of animals can be treated according to the methods of the present invention. In addition to dogs, as disclosed in the above-described exemplary procedure, some non-limiting examples of other animals that can be similarly treated can also include other domestic pets such as cats, as well as horses, cows, sheep, goats, guinea pigs, rats or other bovine, ovine, equine, canine, feline, or rodent species. Those skilled in the art can adjust the treatment procedure taking in account the nature of the animal to be treated.

The following examples are provided to further illustrate the advantages and features of the present invention, but are not intended to limit the scope of the invention.

### EXAMPLE 1

### Preparation and Use of Composition # 1 having Lavendar Scent

Composition # 1 was prepared by mixing, by energetic stirring for about three hours, the following components in the quantities shown below:

(a) about 4.00 mass % of ethoxydiglycol;

(b) about 4.00 mass % of urea;

(c) about 2.00 mass % of cetrimonium chloride;

(d) about 1.00 mass % of sodium lactate;

(e) about 0.50 mass % of galactoarabinan;

(f) about 0.25 mass % of lavender oil lavandua angustifolia;

(g) about 0.20 mass % of sodium lauroyl lactylate;

(h) about 0.10 mass % of poly(ethylene glycol)-120 glucose trioleate;

(i) about 0.10 mass % of cymbopogon schoenanthus extract;

(j) about 0.10 mass % of mentha pulegium extract;

(k) about 0.10 mass % of histidine;

(l) about 0.10 mass % of glycine;

(m) about 0.05 mass % of cedar wood cedrus atlantica bark oil;

(n) about 0.05 mass % of panthenol;

(o) about 0.05 mass % of a blend containing approximately equal amounts of methylchloroisothiazolinone and methylisothiazolinone;

(p) about 0.05 mass % of tea tree melaleuca altermifolia leaf oil;

(q) about 0.02 mass % of allantoin;

(r) about 0.02 mass % of octoxynol-9; and

(s) the balance, water.

The resulting composition # 1 can be applied on a domestic animal in need of improvement of its skin condition, such as a dog or a cat, using the procedure described above and illustrated by FIGS. 1-3.

### EXAMPLE 2

### Preparation and Use of Composition # 2 having Cedar Scent

Composition # 2 was prepared by mixing, by energetic stirring for about three hours, the following components in the quantities shown below:

(a) about 4.00 mass % of ethoxydiglycol;

(b) about 4.00 mass % of urea;

(c) about 2.00 mass % of cetrimonium chloride;

(d) about 1.00 mass % of sodium lactate;

(e) about 0.50 mass % of galactoarabinan;

(f) about 0.50 mass % of pogostemon cablin oil;

(g) about 0.20 mass % of sodium lauroyl lactylate;

(h) about 0.10 mass % of cedar wood cedrus atlantica bark oil;

(i) about 0.10 mass % of poly(ethylene glycol)-120 glucose trioleate;

(j) about 0.10 mass % of mentha pulegium extract;

(k) about 0.10 mass % of histidine;

(l) about 0.10 mass % of glycine;

(m) about 0.07 mass % of octoxynol-9;

(n) about 0.05 mass % of panthenol;

(o) about 0.05 mass % of a blend containing approximately equal amounts of methylchloroisothiazolinone and methylisothiazolinone;

(p) about 0.05 mass % of cymbopogon schoenanthus extract;

(q) about 0.03 mass % of tea tree melaleuca altermifolia leaf oil;

(r) about 0.02 mass % of allantoin;

(s) about 0.01 mass % of peppermint oil mentha piperita;

(t) about 0.01 mass % of spearmint leaf oil menthe viridis; and

(u) the balance, water.

The resulting composition # 2 can be applied on a domestic animal in need of improvement of its skin condition, such as a dog or a cat, using the procedure described above and illustrated by FIGS. 1-3.

### EXAMPLE 3

### Preparation and Use of Composition # 3 (Unscented)

Composition # 3 was prepared by mixing, by energetic stirring for about three hours, the following components in the quantities shown below:

(a) about 4.00 mass % of ethoxydiglycol;

(b) about 4.00 mass % of urea;

(c) about 2.00 mass % of cetrimonium chloride;

(d) about 1.00 mass % of sodium lactate;

(e) about 0.50 mass % of galactoarabinan;

(f) about 0.50 mass % of ordenone fragrance;

(g) about 0.20 mass % of sodium lauroyl lactylate;

(h) about 0.10 mass % of poly(ethylene glycol)-120 glucose trioleate;

(i) about 0.10 mass % of histidine;

(j) about 0.10 mass % of glycine;

(j) about 0.05 mass % of panthenol;

(k) about 0.05 mass % of a blend containing approximately equal amounts of methylchloroisothiazolinone and methylisothiazolinone;

(l) about 0.02 mass % of allantoin;

(m) about 0.02 mass % octoxynol-9; and

(n) the balance, water.

The resulting composition # 3 can be applied on a domestic animal in need of improvement of its skin condition, such as a dog or a cat, using the procedure described above and illustrated by FIGS. 1-3.

Although the invention has been described with reference to the above examples, it will be understood that modifications and variations are encompassed within the spirit and scope of the invention. Accordingly, the invention is limited only by the following claims.

What is claimed is:

1. A method for treating an animal, comprising topically administering to an animal in need thereof an effective amount of an aqueous composition comprising:
(a) a moisturizer;
(b) an anti-itch agent;
(c) a skin conditioner;
(d) a hair conditioner;
(e) a cleansing agent; and
(f) an aromatherapy agent,
thereby treating the animal.

2. The method of claim 1, wherein the composition further comprises a viscosity decreasing agent.

3. The method of claim 2, wherein the viscosity decreasing agent is ethoxydiglycol.

4. The method of claim 1, wherein the topical administering comprises spraying the composition on the skin of the animal.

5. The method of claim 1, wherein the moisturizer is selected from the group consisting of galactoarabinan, urea, sodium lactate, an amino acid, and combinations thereof.

6. The method of claim 5, wherein the amino acid is selected from the group selected from glycine, histidine, and a combination thereof.

7. The method of claim 1, wherein the anti-itch agent is selected from the group consisting of peppermint, tea tree oil, and a combination thereof.

8. The method of claim 1, wherein the skin conditioner is selected from the group consisting of allantoin, pennyroyal, lemongrass, urea, sodium lactate, and combinations thereof.

9. The method of claim 1, wherein the hair conditioner is selected from the group consisting of cetrimonium chloride, sodium lauroyl lactylate, an amino acid, and combinations thereof.

10. The method of claim 9, wherein the amino acid is selected from the group selected from glycine, histidine, and a combination thereof.

11. The method of claim 1, wherein the cleansing agent is selected from a group consisting of poly(ethylene glycol) methyl glucose trioleate and octoxynol-9.

12. The method of claim 1, wherein the aromatherapy agent is selected from the group consisting of lavender, cedar oil, and a combination thereof.

13. The method of claim 1, wherein the composition further comprises an insect repellent.

14. The method of claim 13, wherein the insect repellent is selected from the group consisting of lemongrass, peppermint, pennyroyal, spearmint, patchouli, and combinations thereof, with the further proviso that if lemongrass is used as the skin conditioner, then the group from which the insect repellent is selected does not include lemongrass.

15. The method of claim 1, wherein the animal is selected from a group consisting of a dog, a cat, and a horse.

16. The method of claim 1, wherein the topical administration comprises applying the composition by spraying using a pump or by applying an aerosol by using an aerosol generator, and brushing the composition onto the skin of the animal.

17. An aqueous composition for treating an animal in need of improvement of the skin or hair condition, the composition comprising:
(a) a moisturizer;
(b) an anti-itch agent;
(c) a skin conditioner;
(d) a hair conditioner;
(e) a cleansing agent; and
(f) an aromatherapy agent.

18. The composition of claim 17, wherein the composition further comprises a viscosity decreasing agent.

19. The composition of claim 18, wherein the viscosity decreasing agent is ethoxydiglycol.

20. The composition of claim 17, wherein the moisturizer is selected from the group consisting of galactoarabinan, urea, sodium lactate, an amino acid, and combinations thereof.

21. The composition of claim 20, wherein the amino acid is selected from the group selected from glycine, histidine, and a combination thereof.

22. The composition of claim 17, wherein the anti-itch agent is selected from the group consisting of peppermint, tea tree oil, and a combination thereof.

23. The composition of claim 17, wherein the skin conditioner is selected from the group consisting of allantoin, pennyroyal, lemongrass, urea, sodium lactate, and combinations thereof.

24. The composition of claim 17, wherein the hair conditioner is selected from the group consisting of cetrimonium chloride, sodium lauroyl lactylate, an amino acid, and combinations thereof.

25. The composition of claim 24, wherein the amino acid is selected from the group consisting of glycine, histidine, and a combination thereof.

26. The composition of claim 17, wherein the cleansing agent is selected from the group consisting of poly(ethylene glycol) methyl glucose trioleate and octoxynol-9.

27. The composition of claim 17, wherein the aromatherapy agent is selected from the group consisting of lavender, cedar oil, and a combination thereof.

28. The composition of claim 17, wherein the composition further comprises an insect repellent.

29. The composition of claim 28, wherein the insect repellent is selected from the group consisting of lemongrass, peppermint, pennyroyal, spearmint, patchouli, and combinations thereof, with the further proviso that if lemongrass is used as the skin conditioner, then the group from which the insect repellent is selected does not include lemongrass.

30. The composition of claim 17, wherein the animal is selected from a group consisting of a dog, a cat, and a horse.

31. A kit comprising packaging material and the aqueous composition according to claim 17 contained within the packaging material, wherein the packaging material comprises a label which indicates that the composition can be used for topical administration to an animal in need of improvement of the skin condition.

32. A method for manufacturing the aqueous composition according to claim 17, comprising mixing the components (a) through (f), wherein the composition comprises between about 0.1 and 8.0 mass % of the moisturizer.

33. The method of claim 32, wherein the composition further comprises between about 0.1 and 2.0 mass % of the anti-itch agent.

34. The method of claim 33, wherein the composition further comprises between about 0.5 and 2.5 mass % of the skin conditioner.

35. The method of claim 34, wherein the composition further comprises between about 1.0 and 5.0 mass % of the hair conditioner.

36. The method of claim 35, wherein the composition further comprises between about 0.1 and 0.9 mass % of the cleansing agent.

37. The method of claim 36, wherein the composition further comprises between about 0.01 and 0.9 mass % of the aromatherapy agent.

38. The method of claim 37, wherein the composition further comprises between about 1.0 and 5.0 mass % of a viscosity decreasing agent.

39. The method of claim 37, wherein the viscosity decreasing agent is ethoxydiglycol.

40. The method of claim 37, wherein the composition further comprises between about 0.1 and 0.9 mass % of an insect repellent.

41. The method of claim 40, wherein the insect repellent is selected from the group consisting of lemongrass, peppermint, pennyroyal, spearmint, patchouli, and combinations thereof, with the further proviso that if lemongrass is used as the skin conditioner, then the group from which the insect repellent is selected does not include lemongrass.
